(19) United States
(12) Patent Application Publication
(10) Pub. No.: US 2009/0250626 A1
(43) Pub. Date: Oct. 8, 2009

(54) LIQUID SANITIZATION DEVICE

(75) Inventors: Raoul Schlesser, Raleigh, NC (US); James M. LeMunyon, Oak Hill, VA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Hexatech, Inc.

(21) Appl. No.: 12/418,140

(22) Filed: Apr. 3, 2009

Related U.S. Application Data
(60) Provisional application No. 61/042,474, filed on Apr. 4, 2008.

(51) Int. Cl. H01J 37/20

(52) U.S. Cl. 250/455.11

(57) ABSTRACT

The present invention includes a liquid sanitization device including one or more light emitting diodes (LED) that emit electro-magnetic radiation primarily at two or more distinct wavelengths. These wavelengths should be less than about 300 nm, preferably between about 210 to about 300 nm. The radiation from the light emitting diode or diodes kills or interacts with the DNA or RNA of pathogenic organisms in the liquid to prevent the organisms from reproducing or harming desirable organisms.

### CROSS REFERENCE TO RELATED APPLICATIONS

This application claims the benefit of U.S. Provisional Application No. 61/042,474, filed Apr. 4, 2008, which is incorporated by reference herein in its entirety.

### BACKGROUND OF THE INVENTION

1. Field of the Invention

The present invention relates generally to devices including ultraviolet light emitting diodes for the sterilization of liquids.

2. Description of Related Art

It is known that ultraviolet (“UV”) light within the range of 210 nm to 300 nm, can be used to disinfect liquids such as water, by deactivating the DNA of bacteria, viruses, algae and so forth. Known prior sanitization systems that use UV light typically include a flow-through subsystem, which causes water to travel past an elongated UV light source surrounded by a quartz sleeve and suspended in the flowing water. The quartz sleeve protects the UV light source and its electrical connections from the water while allowing the UV radiation to pass to the water.

The flow-through subsystems include a chamber (i.e., a pipe) that the liquid flows through. The liquid travels past the quartz sleeve, and thus, the UV light source, and is exposed to UV radiation. The UV radiation damages the bacteria, viruses and so forth present in the water. In particular, UV light is effective in destroying certain nucleic acids in microorganisms such that their DNA is disturbed in a manner that causes cell death or prevents reproduction.

Usually, the passing of UV light through a liquid is just one of several techniques utilized in a single sanitization device. For instance filters are generally used to remove particulates from the water while UV light is used to sanitize the water. Further, prior sanitization devices and systems typically rely on mercury-vapor lamps that emit UV light at 254 nm. Such lamps may be high intensity discharge lamps or more commonly low pressure mercury discharge lamps. These lamps are generally chosen because of their mercury line emission properties.

There are several problems associated with using high intensity discharge (HID) or low-pressure discharge lamps for the purpose of sanitizing water. HID sources, for instance, require high voltage and large power sources to operate the lamps. The ballasts for these lamps are large, heavy, and not portable. With these constraints, the HID source may provide an acceptable solution for some industrial settings, but is undesirable and impractical for the home or as a portable unit. Fluorescent lamps that emit UV light often have useful lifetimes of less than one year because the intensity of light output diminishes during the course of time. Such lamps therefore require frequent replacement.

Another problem associated with the use of either low-pressure or high-pressure discharge tubes for the production of ultraviolet radiation is that both of these sources require a significant amount of mercury to produce the desired radiation. Mercury is a significant environmental and health problem. Accordingly, these lamps are often treated as hazardous waste because of the high mercury content.

An additional deficiency of prior liquid sanitization devices is that the UV lamps utilized emit radiation primarily at a single wavelength that may not be tailored to correspond to the maximum spectral sensitivity of a particular microorganism to be killed or damaged. For instance, the commonly used mercury-vapor lamps only emit light at 254 nm. Thus, the use of such lamps are to a varying degree less effective at killing or damaging microorganisms having a wavelength of maximum spectral sensitivity other than 254 nm. Consequently, such systems may provide a relatively inefficient sanitization process for certain types of microorganisms.

Accordingly, there remains a need for a more effective liquid sanitization device and method of sanitizing a liquid without the shortcomings of conventional UV lamps.

### BRIEF SUMMARY OF THE INVENTION

The present invention satisfies at least some of the aforementioned needs by providing a liquid sanitization device including one or more light emitting diodes (LED) that emit electro-magnetic radiation primarily at two or more distinct wavelengths. These wavelengths should be less than about 300 μm, preferably between about 210 to about 280 nm. The radiation from the light emitting diode or diodes interacts with the DNA or RNA of pathogenic organisms in the liquid to prevent the organisms from reproducing or harming desirable organisms. Thus, the device can render various liquid samples safe for consumption or transfusion. Further, embodiments of the present invention can be portable and used away from the power grid. For instance, various embodiments can be used by hikers, campers, and persons living in areas that lack reliable electric power and water sanitization capabilities.

In another aspect, the present invention provides a method for sanitizing a liquid, such as water, where the method includes contacting an unsterilized liquid with a device including one or more light emitting diodes that emit electro-magnetic radiation primarily at two or more distinct wavelengths below about 300 nm, preferably between about 210 to about 280 nm. The electro-magnetic radiation is emitted or directed toward the unsterilized liquid from the light emitting diodes.

### BRIEF DESCRIPTION OF THE DRAWINGS

Having thus described the invention in general terms, reference will now be made to the accompanying drawings, which are not necessarily drawn to scale, and wherein:

FIG. 1 illustrates spectral sensitivity of S6633 spores and MS2 Coliphage relative to LP 254 nm QPB for spores and MS2.

FIG. 2 depicts a sanitization device having multiple LEDs and being mounted on the outside of a conduit carrying a liquid;

FIG. 3 depicts a sanitization device having LEDs located within a UV transparent sleeve; and

FIG. 4 illustrates a hand-held sanitization device.

### DETAILED DESCRIPTION OF THE INVENTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

Pathogenic organisms are life forms that cause human disease. They range in size and complexity, and include molecules like proteinaceous particles (prions); viruses that are visible under an electron microscope; bacteria, fungi, and protozoan parasites that are sometimes visible to the naked eye; and multicellular parasites like tapeworms that may be many meters long. Many live in natural ecosystems, while others are commensal or parasitic on animals and/or humans.

Pathogenic organisms can harm human health in several ways, including consuming nutriment intended for their host (tapeworms); producing poisonous metabolic products (staphylococcus, diphtheria, botulism toxin, and many others); destroying vital organs and tissues (prions, polio, rabies viruses); or interfering with body chemistry (toxic fungus). A few cause cancer (e.g. campylobacter).

UV light has been a known mutagen at the cellular level for more than 100 years, particularly the wavelengths between about 210 to about 280 nm. This range is considered appropriate for germicidal radiation. Accordingly, UV radiation delivered by a mercury-vapor lamp that emits UV with a 254 nanometer wavelength has proven to be beneficial in killing microorganisms such as bacteria (E. coli), viruses (poliovirus, influenza, hepatitis), protozoan cysts (Cryptosporidium, Giardia Lamblia), yeasts and molds.

As used herein, a light emitting diode (“LED”) can include devices comprising one or more light emitting diode structures and an assembly comprising an LED+a phosphor converter, wherein the phosphor converter can comprise one or more phosphor materials. Non-limiting examples include, for example, light emitting p-n junctions and p-i-n diodes.

Certain embodiments of the present invention exhibit improved ability in killing or rendering harmless such pathogenic organisms by including one or more light emitting diodes that emit electro-magnetic radiation primarily at two or more distinct wavelengths. By providing UV light in multiple distinct wavelengths less than about 300 nm, preferably between about 210 to about 280 nm or alternatively from about 260 to about 280 nm, the germicidal maximum spectral sensitivity wavelength region is more thoroughly covered. As such, the radiation from the light emitting diode or diodes either kills the pathogenic organisms or interacts with the DNA or RNA of pathogenic organisms in the liquid to prevent the organisms from reproducing or harming desirable organisms.

As understood by one skilled in the art, the emission of light primarily at a given wavelength, for example at 270 nm, will also include light output, although to substantially lesser degree, at surrounding wavelengths. Thus, for example only, if an LED emits light primarily at 270 nm, it should be understood that the LED will also emit some light at surrounding wavelengths, such as from 268 to 272. This spectral bandwidth around the primary or central wavelength is typically characterized by its full-width, half-maximum (FWHM). FWHM is simply an expression of the extent of a function, given by the difference between the two extreme values of the independent variable (e.g., wavelength) at which the dependent variable (e.g., spectral intensity) is equal to half of its maximum value. In certain embodiments, the FWHM can comprise about 5% to about 7% of the central wavelength. In one embodiment the spectral emission may comprise about a 10 to 12 nm spectral bandwidth (FWHM). However, the light output at the outer wavelengths is greatly reduced compared to the primary wavelength (e.g., 270 nm in our example). Thus, emission of light primarily at a given wavelength corresponds to a peak emission, wherein the peak emission can be either a maximum peak emission or a local peak emission. Beneficially, however, the bandwidths of LEDs exhibit tight bandwidths. Thus, the use of LEDs allows for increased precision in administering UV light at specific wavelengths in which various pathogenic organisms absorb radiation to the greatest degree. For example, if a maximum spectral sensitivity of radiation for a given organism is 275 nm, then a UV LED can be constructed to emit UV light primarily at this wavelength to improve the efficiency in sterilizing liquids containing such an organism.

In one embodiment of the present invention, the liquid sanitization device can deliver a dose greater than 40 mJ/cm² throughout the functional life of the LED in accordance with public health recommendations and regulations. In particular, the current standard for Class A systems for UV water treatment, namely NSF/ANSI Standard 55, mandates that such systems provide at least 40 mJ/cm². While Class A systems are supposed to disinfect water to a safe level for consumption, Class B systems are qualified as supplemental sanitization systems for drinking water that already complies with local health standards. Class B systems are required to provide at least 16 mJ/cm². Embodiments of the present invention can deliver a dose at the end of the LED life, depending on the particular application for the device, ranging from about 5 to about 100 mJ/cm², or about 15 to about 85 mJ/cm², or about 20 to about 85 mJ/cm², or about 30 to about 60 mJ/cm², or about 35 to about 50 mJ/cm². In one alternative embodiment, the device can deliver a dose throughout the time of its use in sterilization from about 10 to about 30 mJ/cm², or about 12 to about 25 mJ/cm², or about 14 to about 20 mJ/cm². In yet another alternative embodiment, the device can deliver from about 80 to about 110 mJ/cm², or about 80 to about 105 mJ/cm², or about 95 to about 105 mJ/cm². As such, sanitization devices according to embodiments of the present invention emit radiation that can interact with the RNA or DNA of a multitude of micro-organisms. A non-limiting list of such micro-organisms is provided in Table I. The bottom of Table 1 provides the list of sources from which the UV dose values were obtained. Also, the numbers following each micro-organism indicate which specific source or sources from which the UV dose value for each micro-organism was obtained.

TABLE I
Microorganisms deactivated by germicidal ultraviolet light

| | UVDose |
|---|---|
| | nJ/cm² |
| Bacteria | |
| Agrobacterium lumefaciens 5 | 8,500 |
| Bacillus anthracis 1, 4, 5, 7, 9 (anthrax veg.) | 8,700 |
| Bacillus anthracis Spores (anthrax spores)* | 46,200 |
| Bacillus megatherium Sp. (veg) 4, 5, 9 | 2,500 |
| Bacillus megatherium Sp. (spores) 4, 9 | 5,200 |
| Bacillus paratyphosus 4, 9 | 6,100 |
| Bacillus subtilis 3, 4, 5, 6, 9 | 11,000 |
| Bacillus subtilis Spores 2, 3, 4, 6, 9 | 22,000 |
| Clostridium tetani | 23,100 |
| Clostridium botulinum | 11,200 |
| Corynebacterium diphtheriae 1, 4, 5, 7, 8, 9 | 6,500 |
| Dysentery bacilli 3, 4, 7, 9 | 4,200 |
| Eberthella typhosa 1, 4, 9 | 4,100 |
| Escherichia coli 1, 2, 3, 4, 9 | 6,600 |
| Legionella bozemanii 5 | 3,500 |
| Legionella dumoffill 5 | 5,500 |
| Legionella gormanil 5 | 4,900 |
| Legionella micdadei 5 | 3,100 |
| Legionella longbeachae 5 | 2,900 |
| Legionella pneumophila (Legionnaire's Disease) | 12,300 |
| Leptospira canicola-Infectious Jaundice 1, 9 | 6,000 |
| Leptospira interrogans 1, 5, 9 | 6,000 |
| Micrococcus candidus 4, 9 | 12,300 |
| Micrococcus sphaeroides 1, 4, 6, 9 | 15,400 |
| Mycobacterium tuberculosis 1, 3, 4, 5, 7, 8, 9 | 10,000 |
| Neisseria catarrhalis 1, 4, 5, 9 | 8,500 |
| Phytomonas tumefaciens 1, 4, 9 | 8,500 |
| Proteus vulgaris 1, 4, 5, 9 | 6,600 |
| Pseudomonas aeruginosa (Environ. Strain) 1, 2, 3, 4, 5, 9 | 10,500 |
| Pseudomonas aeruginosa (Lab. Strain) 5, 7 | 3,900 |
| Pseudomonas fluorescens 4, 9 | 6,600 |
| Rhodospiμrillum rubrum 5 | 6,200 |
| Salmonella enteritidis 3, 4, 5, 9 | 7,600 |
| Salmonella paratyphi (Enteric Fever) 5, 7 | 6,100 |
| Salmonella Species 4, 7, 9 | 15,200 |
| Salmonella typhimurium 4, 5, 9 | 15,200 |
| Salmonella typhi (Typhoid Fever) 7 | 7,000 |
| Salmonella | 10,500 |
| Sarcina lutea 1, 4, 5, 6, 9 | 26,400 |
| Serratia marcescens 1, 4, 6, 9 | 6,160 |
| Shigella dysenteriae - Dysentery 1, 5, 7, 9 | 4,200 |
| Shigella flexneri - Dysentery 5, 7 | 3,400 |
| Shigella paradysenteriae 4, 9 | 3,400 |
| Shigella sonnei 5 | 7,000 |
| Spirillum rubrum 1, 4, 6, 9 | 6,160 |
| Staphylococcus albus 1, 6, 9 | 5,720 |
| Staphylococcus aureus 3, 4, 6, 9 | 6,600 |
| Staphylococcus epidermidis 5, 7 | 5,800 |
| Streptococcus faecaila 5, 7, 8 | 10,000 |
| Streptococcus hemolyticus 1, 3, 4, 5, 6, 9 | 5,500 |
| Streptococcus lactis 1, 3, 4, 5, 6 | 8,800 |
| Streptococcus pyrogenes | 4,200 |
| Streptococcus salivarius | 4,200 |
| Streptococcus viridans 3, 4, 5, 9 | 3,800 |
| Vibrio comma (Cholera) 3, 7 | 6,500 |
| Vibrio cholerae 1, 5, 8, 9 | 6,500 |
| Molds | |
| Aspergillus amstelodami | 77,000 |
| Aspergillus flavus 1, 4, 5, 6, 9 | 99,000 |
| Aspergillus glaucus 4, 5, 6, 9 | 88,000 |
| Aspergillus niger (breed mold) 2, 3, 4, 5, 6, 9 | 330,000 |
| Mucor mucedo | 77,000 |
| Mucor racemosus (A & B) 1, 3, 4, 6, 9 | 35,200 |
| Oospora lactis 1, 3, 4, 6, 9 | 11,000 |
| Penicillium chrysogenum | 56,000 |
| Penicillium digitatum 4, 5, 6, 9 | 88,000 |
| Penicillium expansum 1, 4, 5, 6, 9 | 22,000 |
| Penicillium roqueforti 1, 2, 3, 4, 5, 6 | 26,400 |
| Rhizopus nigricans (cheese mold) 3, 4, 5, 6, 9 | 220,000 |
| Protozoa | |
| Chlorella vulgaris (algae) 1, 2, 3, 4, 5, 9 | 22,000 |
| Blue-green Algae | 420,000 |
| E. hystolytica | 84,000 |
| Giardia lamblia (cysts) 3 | 100,000 |
| Nematode Eggs 6 | 40,000 |
| Paramecium 1, 2, 3, 4, 5, 6, 9 | 200,000 |
| Virus | |
| Adeno Virus Type III 3 | 4,500 |
| Bacteriophage 1, 3, 4, 5, 6, 9 | 6,600 |
| Coxsackie | 6,300 |
| Infectious Hepatitis 1, 5, 7, 9 | 8,000 |
| Influenza 1, 2, 3, 4, 5, 7, 9 | 6,600 |
| Rotavirus 5 | 24,000 |
| Tobacco Mosaic 2, 4, 5, 6, 9 | 440,000 |
| Yeasts | |
| Baker's Yeast 1, 3, 4, 5, 6, 7, 9 | 8,800 |
| Brewer's Yeast 1, 2, 3, 4, 5, 6, 9 | 6,600 |
| Common Yeast Cake 1, 4, 5, 6, 9 | 13,200 |
| Saccharomyces cerevisiae 4, 6, 9 | 13,200 |
| Saccharomyces ellipsoideus 4, 5, 6, 9 | 13,200 |
| Saccharomyces sp. 2, 3, 4, 5, 6, 9 | 17,600 |

1 “The Use of Ultraviolet Light for Microbial Control”, Ultrapure Water, April 1989.
2 William V. Collentro, “Treatment of Water with Ultraviolet Light - Part I”, Ultrapure Water, July/August 1986.
3 James E. Cruver, Ph.D., “Spotlight on Ultraviolet Disinfection”, Water Technology, June 1984.
4 Dr. Robert W. Legan, “Alternative Disinfection Methods-A Comparison of UV and Ozone”, Industrial Water Engineering, March/April 1982.
5 Unknown
6 Rudolph Nagy, Research Report BL-R-6-1059-3023-1, Westinghouse Electric Corporation.
7 Myron Lupal, “UV Offers Reliable Disinfection”, Water Conditioning & Purification, November 1993.
8 John Treij, “Ultraviolet Technology”, Water Conditioning & Purification, December 1995.
9 Bak Srikanth, “The Basic Benefits of Ultraviolet Technology”, Water Conditioning & Purification, December 1995
*Approximate - Various sources may report different inactivation dosages.

According to embodiments of the present invention, one or more LEDs can emit multiple wavelengths ranging from about 210 to about 300 nm. By way of example only, a single LED (e.g., a single diode) can be used in which the LED emits radiation primarily at 250 nm and also primarily at 260 nm. Alternatively, embodiments of the present invention can include multiple LEDs (i.e., more than one diode), wherein each LED emits radiation at different and distinct wavelengths to more completely irradiate a liquid in a region ranging from about 210 to about 300 nm. In other embodiments, the sanitization device can include multiple LEDs wherein each LED, or group of LEDs which can be configured into an array, emits radiation at a single and different wavelength. In various embodiments, phosphor conversion technology can be employed. U.S. Published Appl. No. 2007/0159067, which is incorporated by reference herein, generally describes an application of phosphor conversion; wherein the LED device emits light at multiple wavelengths such as white light, blue-green light, or pink light, using blue LED chips or ultraviolet LED chips. Although the particular devices described in U.S. Published Appl. No. 2007/0159067 are not suitable for water sanitization, a similar approach of phosphor conversion can be used for the sanitization of water. As such, in one embodiment the LEDs can each comprise an LED+a phosphor converter, which can include one or more phosphorescing materials to convert light generated into longer wavelengths. In one embodiment, deep UV light (e.g., less than 240 nm) can be converted into multiple longer wavelengths (e.g., greater than 240 nm). UV LEDs are commercially available from Sensor Electronic Technology, Inc. (Columbia, S.C.).

In one preferred embodiment, the wavelengths at which radiation can be emitted from one or more LEDs can range from about 250 to about 300 nm, or from about 255 to about 290 nm. In more preferable embodiments, the liquid sanitization device includes an LED or LEDs that can emit radiation from about 260 to about 285 nm, from about 260 to about 280 nm, or from about 265 to about 275 nm.

In certain exemplary embodiments, the invention provides a sanitization system comprising a first LED (or array of LEDs) having a first primary wavelength of between about 210 and about 300 nm and a second LED (or array of LEDs) having a second primary wavelength between about 210 and about 300 nm that is different from the first primary wavelength. The first primary wavelength could be between about 210 to about 250 nm (e.g., 210, 220, 230, 240, or 250 nm) and the second wavelength could be between about 260 nm and about 300 nm (e.g., 260, 270, 280, 290, or 300 nm). Alternatively, one or more additional LEDs (or arrays of LEDs) could be added to the system, each additional LED emitting at another distinct primary wavelength between about 210 and about 300 nm. The system could also include multiple LEDs emitting at each primary wavelength.

Examples of commercially available ultraviolet light emitting diodes, for example, include the UVTOP® line of UV LEDs provided by Sensor Electronic Technology (Columbia, S.C.) that include LEDs exhibiting a peak emission down to about 250 nm. Additional commercially available UV LEDs include Seoul Optodevice Company's (Seoul, South Korea) BioUV 280 nm series, their 265 nm series and their 255 nm series.

In one preferred embodiment of the present invention, the LEDs are produced from aluminum nitride-based materials or alternatively from aluminum gallium nitride-based materials. Ultraviolet LEDs produced from these materials have been known to emit radiation down to about 210 nm.

According to one embodiment of the present invention, a liquid sanitization device can include a single LED that emits electro-magnetic radiation primarily at two or more distinct wavelengths being less than about 300 nm. Each of the distinct wavelengths can comprise any wavelength between about 210 to about 300 nm or any intermediate range described herein. By way of example, the LED according to one embodiment can emit radiation primarily at 250 nm and also at 270 nm, or alternatively at 250 nm, 260 nm, and 270 nm. Although the device can be embodied in numerous configurations, the UV light is emitted generally towards the liquid to be sanitized or disinfected as generally known in the art. Accordingly, the radiation interacts with the DNA or RNA of pathogenic organisms in a liquid exposed to the radiation and either kills or prevents the organisms from reproducing or harming desirable organisms, such as mammals.

According to another embodiment of the present invention, a liquid sanitization device can include multiple LEDs that each emit electro-magnetic radiation primarily at two or more distinct wavelengths being less than about 300 nm. Each of the distinct wavelengths can comprise any wavelength between about 210 to about 300 nm or any intermediate range described herein.

According to yet another embodiment of the present invention, a liquid sanitization device can include multiple LEDs, within a single arrangement, that each emit electro-magnetic radiation primarily at one distinct and different wavelength being less than about 300 nm, preferably between about 210 to about 300 nm. Each of the LEDs can emit radiation of wavelength comprising any wavelength between about 210 to about 300 nm or any intermediate range described herein. Alternatively, embodiments of the invention can include multiple groups of LEDs, wherein each group includes multiple LEDs. Each LED within a first group can emit radiation primarily at the same wavelength, while each LED with in a second group can emit radiation primarily at a same wavelength being different from the wavelength emitted by the first group. In preferred embodiments, each group of LEDs emit radiation primarily at a wavelength that corresponds to the maximum spectral sensitivity of a particular pathogenic organism. Thus, each group can specifically target a different organism for irradiating. Depending on the known organisms in a liquid, numerous groups of specifically tailored LEDs can be incorporated into a single device. As such, the radiation interacts with the DNA or RNA of any pathogenic organisms in a liquid exposed to the radiation and either kills or prevents the organisms from reproducing or harming desirable organisms, such as mammals.

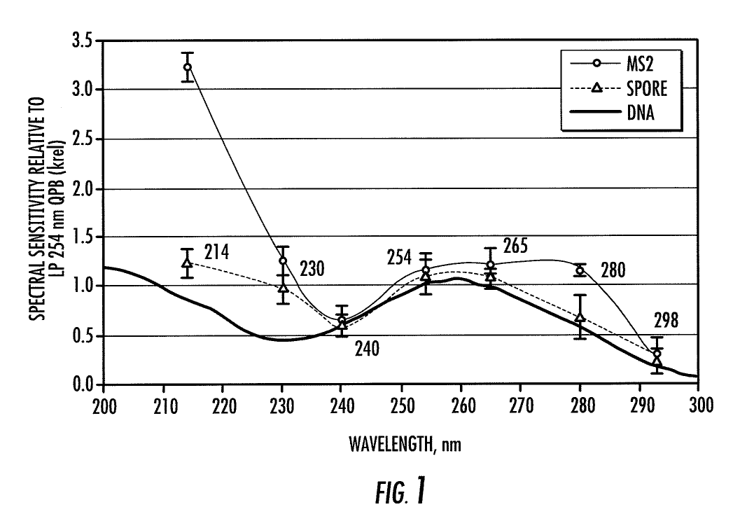

Additional embodiments of the present invention comprise a liquid sanitization device including a single LED that is tailored to emit radiation primarily at two or more wavelengths that correspond to the maximum spectral sensitivity of pathogenic organisms in a liquid for treatment. As such, the radiation emitted at each specific wavelength more efficiently interacts with the DNA or RNA of specific pathogenic organisms in a liquid exposed to the radiation to either kill or prevent the organisms from reproducing or harming desirable organisms. In one alternative embodiment, a liquid sanitization device can include multiple LEDs that have been tailored to emit electro-magnetic radiation primarily at two or more distinct wavelengths that correspond to the maximum spectral sensitivity of a pathogenic organism or organisms. In some instances, the nature of contaminating micro-organisms remains unknown and may vary over time. Thus, embodiments of the present invention more thoroughly or completely cover of a wider wavelength band (e.g., 240-280 nm) to more safely remove or deactivate any kind of micro-organisms. Accordingly, such embodiments can more thoroughly and efficiently sanitize a liquid containing a variety of micro-organisms because different organisms have different UV sensitivity spectra. For instance, Linden et al (2005), Environ. Sci. Technol., Spectral Sensitivity of Bacillus subtilis Spores and MS2 Coliphage for Validation Testing of Ultraviolet Reactors for Water Disinfection: 39, 7845-7852, discuss differences in spectral sensitivity for bacillus subtilis spores and MS2 Coliphage. In particular, Linden et al (2005) illustrates that the spectral sensitivity of at least some organisms deviates from the DNA spectral sensitivity spectrum. In fact, bacillus subtilis is about twice as sensitive to 280 nm radiation than expected based on DNA absorbance. FIG. 1 [Linden et al (2005)], illustrates that, on a relative basis, MS2 is most sensitive to wavelengths below 230 nm and bacillus subtilis spores are most sensitive to wavelengths around 265 nm. Further, the efficiency of MS2 inactivation at 214 nm is about three times higher compared to that of MS2 at 254 nm.

In various embodiments, the sanitization device can be part of a flow-through subsystem where the liquid, such as water or mammalian blood, travels through an elongated conduit. The sanitization device can be mounted external to the conduit wherein the conduit includes an ultraviolet transparent segment for allowing the radiation from the LED(s) to treat the liquid flowing through the conduit. Alternatively the sanitization device can be operatively connected to an ultraviolet transparent sleeve, such as a quartz sleeve or the like, so that as the liquid travels past the quartz sleeve, the liquid is exposed to the UV radiation from the sanitization device. The quartz sleeve protects the sanitization device and its electrical connections from the liquid while allowing the UV radiation to pass to the liquid. In one embodiment, the quartz sleeve is suspended in a conduit of flowing liquid.

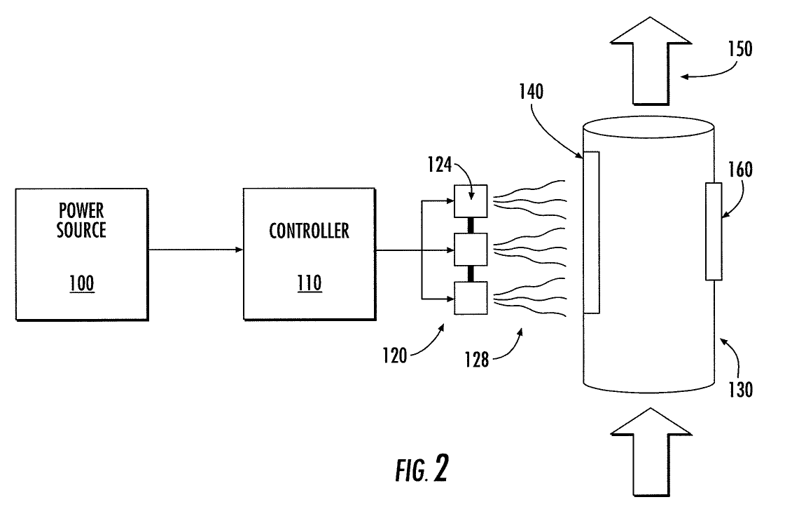

FIG. 2 illustrates a sanitization device according to one embodiment of the present invention. In this particular embodiment, multiple LEDs 124 are provided in a single array 120 which is externally located from a conduit 130 carrying liquid 150 for treatment. The conduit includes a UV transparent segment 140 proximately located to the LEDs such that when the LEDs are provided power from a power source 100, the radiation 128 emitted from the LEDs passes into the flowing liquid within the conduit. Optionally, a meter 160 for measuring the radiation received by the liquid can be included either internally or externally to the conduit. Additionally, a controller 110 can optionally be included. The controller can include various hardware, software, switches and timing circuits as is known in the art. In one embodiment the controller can include an on/off switch and a timing circuit that turns the LEDs off after a predetermined time. In another embodiment, the meter 160 for measuring the radiation received from the LEDs can be operatively connected to the controller such that upon indication by the meter that the liquid has received a predetermined amount of radiation, the controller turns the LEDs off.

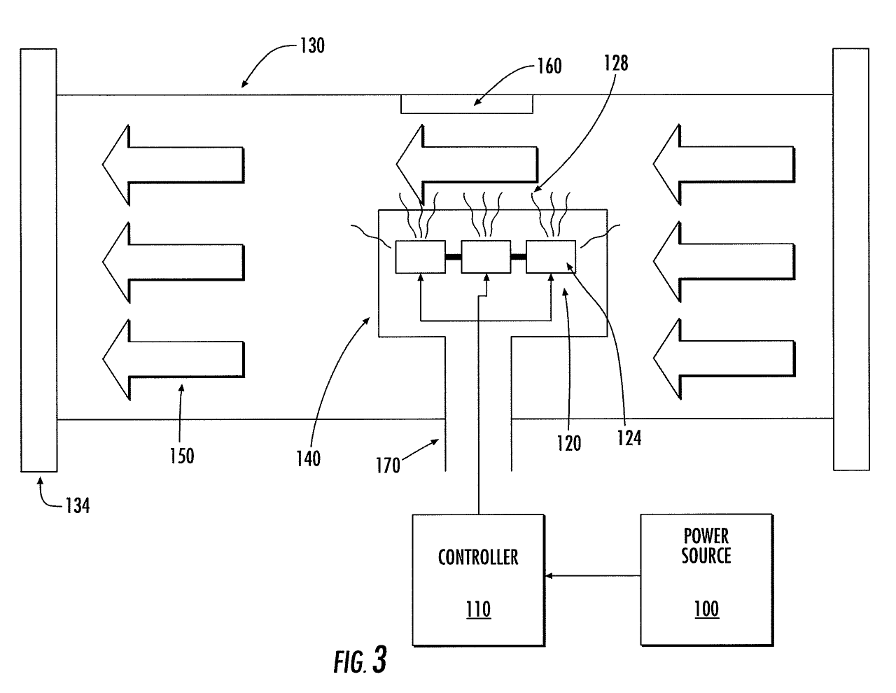

FIG. 3 illustrates a sanitization device according to one embodiment of the present invention. In this particular embodiment, multiple LEDs 124, provided in a single array 120, are located within a UV transparent sleeve 140 such that all electrical components are protected from liquid within a conduit 130 having liquid 150 flowing therethrough. The UV transparent sleeve containing the array of LEDs can be suspended within the conduit by an access port 170. The access port also allows for electrical connection of the LEDs to a controller 110, which can be optionally provided, or directly to a power source 100. In one embodiment, a meter 160 for measuring the radiation received by the liquid can be included either internally or externally to the conduit. In one embodiment, a flow meter (not shown) or the like can by included to provide indication of when liquid is flowing through the pipe. The optional controller can include various hardware, software, switches and timing circuits as is known in the art. In one embodiment, the conduit can include flanges 134. In such embodiments, a flanged conduit including the sanitization device can by packaged and sold as a single unit. Beneficially, the flanged conduit including the sanitization device can be easily incorporated into existing piping systems.

The UV radiation damages the DNA or RNA of the pathogenic organisms such that they no longer have the ability to reproduce and multiply. In such embodiments, the liquid can either be pumped through the conduit for treatment or simply allowed to flow through the conduit due to gravitational forces. Further, the device can be turned on under the control of one or more switches that are, in turn, under the control of a liquid sensor that senses when liquid is within the conduit for treatment. Such sensors are well known in the art. As just one example, the sensor can comprise a flow meter. If desired, the device can also include a timing circuit that turns the LEDs off after a predetermined time. In one embodiment, a battery powers the various components of the device while in another embodiment the device can be solar powered. In an alternative embodiment, the device also includes an electrical power storage device.

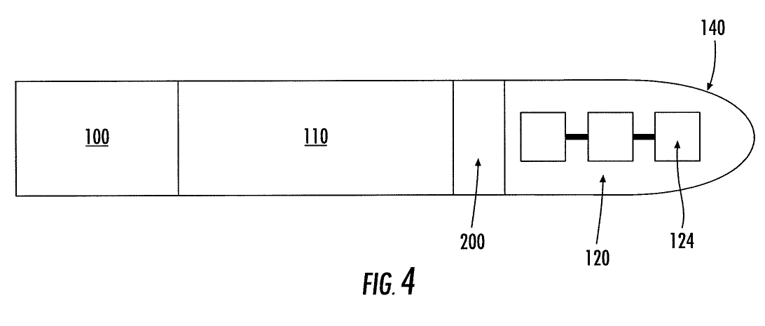

In one alternative embodiment according to the present invention, the sanitization device can comprise a hand-held device for sanitizing small containers of water, such as cups and thermoses. As illustrated in FIG. 4, the hand-held liquid sanitization device can include an outwardly-extending pen-light sized configuration of solid state devices (i.e., UV LEDs) 124, optionally provided in an array 120, that emit ultraviolet light in the range from about 210 to about 300 nm (or in any intermediate range described herein). The LEDs are housed within a UV transparent sleeve 140, wherein the sleeve can take many forms such as a cap. The UV transparent sleeve allows radiation to pass from the LEDs into a liquid for treatment surrounding the device. The device can be powered on and off using a controller 110, which can include any combination of one or more switches, hardware, and software. The device can also optionally include a liquid-level sensor 200 in communication with the controller 110 that senses when the LED array 120 is immersed in liquid. If desired, the controller can also include a timing circuit that turns the LEDs off after a predetermined time. In one embodiment, a power source 100, such as a battery, powers the various components of the device while in another embodiment the device can be solar powered. In an alternative embodiment, the device also includes an electrical power storage device (not shown).

Certain embodiments of the present invention provide a more efficient treatment of a liquid, such as water or alternatively mammalian blood, because the solid state device or devices (i.e., LED(s)) can be tailored such that the emission of radiation corresponds to the maximum spectral sensitivity of particular organisms. As such, the time and energy required to kill pathogenic organisms or render them harmless is reduced. Accordingly, more liquid can be treated in a given period of time. Thus, embodiments of the present invention provide a higher throughput for treatment of various liquids. Further, by targeting organisms at their wavelength of maximum spectral sensitivity the quality of the treated liquid is improved. For instance, prior art devices kill most pathogenic organisms in water through UV light, but may fall short of killing or debilitating them to satisfy the EPA's standard for drinking water. By emitting radiation at more than one wavelength, embodiments of the present invention more thoroughly destroy such undesirable organisms in the UV range.

According to various embodiments of the present invention, the liquid sanitization device can advantageously be incorporated into traditional water purification systems for removing contaminants from water. Such water purification systems can be designed based on the needs of the particular application, and can be adapted for purification of drinking water, as well as purification of liquids in medical, laboratory, and industrial settings. As such, embodiments of the present invention can by used in conjunction with other components or methods typically found in water purification systems such as filtration, water softening, reverse osmosis, ultrafiltration, molecular stripping, deionization, and carbon treatment. These additional water purification techniques may be needed to remove hydrocarbons, particulate sand, suspended particles of organic material, minerals, toxic metals (e.g., lead, copper, chromium), and the like.

Additionally, various embodiments can by easily powered by any traditional power source. Since the device employs LEDs as the UV source, the power requirement is greatly reduced. In one embodiment, the sanitization device is lightweight and portable such that it can be carried by hikers and campers.

In certain embodiments, a sanitization device can include a measurement device for measuring the irradiance received by the liquid from the LED or LEDs. In one embodiment the measurement device can comprise a commercially available UV detector. Such detectors measure the radiant flux that passes from the LED(s), preferable through the maximum depth of liquid to be treated. A UV detector can send a signal proportional to the radiant flux it receives. In the event that the LED(s) emit less light than required, or if the liquid is too dirty, the detector signal can drop below a pre-defined threshold and trigger an alarm. One such example of a suitable UV detector includes UV 10.SF—Ultraviolet Detector provided from PerkinElmer® (Fremont, Calif.).

Another aspect of the present invention comprises a method for sanitizing a liquid, such as water or mammalian blood; where the method includes contacting an unsterilized liquid with a device including one or more LEDs that emit electro-magnetic radiation primarily at two or more distinct wavelengths below about 300 nm, preferably between about 260 to about 280 rm. Alternatively, the LED-containing device can be housed exterior to the liquid-containing compartment such that the liquid does not contact the device. In either case, the electro-magnetic radiation is emitted or directed toward the unsterilized liquid from the light emitting diodes.

In one embodiment, the liquid in need of disinfecting or sanitization can be either pumped or allowed to flow through a conduit due to gravitational forces. While flowing through the conduit, the liquid is exposed to UV light at primarily two or more distinct wavelengths. The UV light can be emitted by one or more LEDs that can be housed within a single sanitization device. In one embodiment, the liquid can be recirculated through the conduit for repeated exposure to the UV light. The electro-magnetic radiation emitted from the LED or LEDs can be directed toward the unsterilized liquid for a predetermined time to ensure that the radiation kills or sufficiently interacts with the DNA or RNA of pathogenic organisms in the liquid to prevent the organisms from reproducing or harming desirable organisms. Further, a meter can be used to measure the irradiance received by the liquid from the LED or LEDs.

In another embodiment according to the present invention, a liquid in need of disinfecting or sanitization due to inclusion of pathogenic organisms can be contacted with a hand-held sanitization device as described herein. The hand-held device can be immersed in a container of unsterilized liquid, such that the UV LED or LEDs are immersed in the untreated liquid. In one embodiment, the device includes a meter or probe for sensing that the UV source is immersed fully in the unsterilized water. After immersion, the UV source can be turned on to emit ultraviolet radiation primarily at two or more distinct wavelengths into the batch of unsterilized liquid. The electro-magnetic radiation emitted from the LED or LEDs can be directed toward the unsterilized liquid for a predetermined time to ensure that the radiation kills or sufficiently interacts with the DNA or RNA of pathogenic organisms in the liquid to prevent the organisms from reproducing or harming desirable organisms.

In certain embodiments, various liquids including one or more pathogenic organisms can be purified by emitting UV light into the liquid at two or more distinct wavelengths, where at least two of the distinct wavelengths are from about 210 to about 300 nm. Most preferably, at least two of the distinct wavelengths emitted by the LED or LEDs correspond to a wavelength of maximum spectral sensitivity for one or more pathogenic organisms. For instance, in one embodiment a first distinct wavelength corresponds to a wavelength of maximum spectral sensitivity for a first pathogenic organism and a second distinct wavelength corresponds to a wavelength of maximum spectral sensitivity for a second pathogenic organism. As the number of pathogenic organisms targeted increases, the number or separate and distinct wavelengths can be increased to correspond to each pathogenic organism.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A liquid sanitization device comprising one or more light emitting diodes that emit electro-magnetic radiation primarily at two or more distinct wavelengths less than about 300 nm, such that the radiation interacts with DNA or RNA of pathogenic organisms in a liquid to prevent the organisms from reproducing or harming desirable organisms.

2. The device of claim 1, wherein the device emits the radiation generally toward the liquid.

3. The device of claim 1, further comprising a measurement device for measuring irradiance received by the liquid from the light emitting diodes.

4. The device of claim 1, further comprising a solar energy converter so that the light emitting diodes can be powered by solar power.

5. The device of claim 1, further comprising an electrical power storage device.

6. The device of claim 1, wherein the light emitting diodes comprise aluminum nitride-based materials.

7. The device of claim 1, wherein the light emitting diodes comprise aluminum gallium nitride-based materials.

8. The device of claim 1, wherein multiple light emitting diodes are arranged in a single array.

9. The device of claim 1, wherein at least one of the distinct wavelengths comprises a wavelength that corresponds to a wavelength of maximum spectral sensitivity for a pathogenic organism.

10. The device of claim 1, wherein at least two of the distinct wavelengths correspond to a wavelength of maximum spectral sensitivity for a first pathogenic organism having a first maximum spectral sensitivity wavelength and a second pathogenic organism having a second maximum spectral sensitivity wavelength.

11. The device of claim 1, wherein the liquid comprises water.

12. The device of claim 1, wherein the liquid comprises mammalian blood.

13. A method of sanitizing an unsterilized liquid, comprising:
(a) providing a device including one or more light emitting diodes that emit electro-magnetic radiation primarily at two or more distinct wavelengths below about 300 nm; and
(b) emitting electro-magnetic radiation toward the unsterilized liquid from the light emitting diodes primarily at two or more distinct wavelengths below about 300 nm.

14. The method of claim 13, wherein the unsterilized liquid comprises water.

15. The method of claim 13, wherein the unsterilized liquid comprises mammalian blood.

16. The method of claim 13, wherein the unsterilized liquid flows past the light emitting diodes in a conduit.

17. The method of claim 13, wherein the device including one or more light emitting diodes is immersed in the unsterilized liquid.

18. The method of claim 13, wherein emitting electro-magnetic radiation toward the unsterilized liquid occurs for a predetermined period of time such that the radiation sufficiently interacts with DNA or RNA of pathogenic organisms in the liquid sufficient to prevent the organisms from reproducing or harming desirable organisms.

19. The method of claim 13, wherein at least one of the distinct wavelengths comprises a wavelength that corresponds to a wavelength of maximum spectral sensitivity for a pathogenic organism.

20. The method of claim 13, wherein at least two of the distinct wavelengths correspond to a wavelength of maximum spectral sensitivity for a first pathogenic organism having a first maximum spectral sensitivity wavelength and a second pathogenic organism having a second maximum spectral sensitivity wavelength.

21. A liquid sanitization system, comprising;
(a) a device housing at least one ultraviolet light emitting diode, wherein the at least one light emitting diode emits radiation at two or more distinct wavelengths from about 250 nm to about 280 nm; and
(b) a power source for powering the at least one ultraviolet light emitting diode such that when power is supplied to the at least one ultraviolet light emitting diode, the radiation therefrom interacts with DNA or RNA of pathogenic organisms in a liquid to prevent the organisms from reproducing or harming desirable organisms.

22. The system of claim 21, further comprising a quartz sleeve operatively connected to the at least one light emitting diode such that liquid flowing past the quartz sleeve or surrounding the quartz sleeve is exposed to radiation.

23. The system of claim 21, wherein the quartz sleeve is suspended in a conduit of flowing water.
